(19) United States
(12) Patent Application Publication
(10) Pub. No.: US 2017/0157676 A1
(43) Pub. Date: Jun. 8, 2017

(54) BIOCOMPATIBLE COLLOIDAL SOLUTION OF SILVER NANOPARTICLES IN NON-AQUEOUS POLAR SOLVENT AND METHOD OF OBTAINING THEREOF

(71) Applicant: TOVARISTVO Z OBMEZHENOU VIDPOVIDALNISTU "NANOMEDTRAST", Kyiv (UA)

(72) Inventors: Oleksandra Evgenivna RAIEVS'KA, Kyiv (UA); Oleksandr Leonidovych STROIUK, Kyiv (UA)

(73) Assignee: TOVARISTVO Z OBMEZHENOU VIDPOVIDALNISTU "NANOMEDTRAST", Kyiv (UA)

(21) Appl. No.: 15/324,021

(22) PCT Filed: Jul. 8, 2015
(86) PCT No.: PCT/UA2015/000058

§ 371 Date: Jan. 5, 2017

(30) Foreign Application Priority Data
Jul. 8, 2014 (UA) A 2014 07694

(51) Int. Cl. B22F 9/24; A61K 9/16; A61K 33/38; A61K 8/02; A61K 8/19; A61K 8/46; B22F 1/00; A61K 9/08

(52) U.S. Cl. CPC B22F 9/24; B22F 1/0022; B22F 1/0074; A61K 9/1617; A61K 9/08; A61K 8/0241; A61K 8/19; A61K 8/46; A61K 33/38; B22F 2009/245; B22F 2301/255; B22F 2304/054; B22F 2998/10

(57) ABSTRACT

The present application relates to colloidal chemistry, specifically to methods of synthesising silver nanoparticle colloids in a non-aqueous solvent, preferably, in dimethyl sulfoxide. In particular these silver nanoparticles have an average size of 12-20 nm and are in a biocompatible colloidal solution.

The invention relates to colloidal chemistry, specifically to methods of synthesising silver nanoparticle (NP) colloids in a non-aqueous solvent, and may be used, in particular, in the pharmaceutical or cosmetic industry, e.g. to produce silver nanoparticle colloids in a form suitable for introduction of silver nanoparticles and silver into soft dosage forms and cosmetic products—ointments, creams, etc.

The following colloidal solutions of silver nanoparticles, containing metallic silver in nanodispersed state stabilized with non-toxic products, are known to the applicant.

In particular, the method of synthesising silver nanoparticles and colloidal solution of silver nanoparticles, so produced, with synthetic humic acids is known in the prior art. The method comprises synthesising silver nanoparticles by reconstituting soluble silver salts derived with synthetic humic acids in an alkaline medium (Ukrainian Patent No. 94989, Publication No. 12, 2011, IPC: A61K 33/38, C08G 8/00).

Further, the prior art discloses the method of obtaining a colloidal solution of metal nanoparticles, comprising dissolving gold iodide or silver iodide in water or a non-aqueous solvent, blowing gaseous carbon oxide (II) through the solution, followed by heating the solution up to 50° C., or adding an organic liquid, which does not mix with water or an non-aqueous solvent. Carbon tetrachloride (Freon-10, Khladon-10), CCl₄, in the amount up to 0.1 of the volume of the resulting solution, may be used as organic liquid (Patent RU 2357797 C2, IPC publications: B01J13/00, B82B1/00, C01G5/00, C01G7/00 (2006.01)).

The resulting colloidal solution is sufficiently pure as it is free from anion salt impurities.

The method of obtaining a colloidal solution of silver nanoparticles and a colloidal solution of silver nanoparticles with quite stable distribution by solvodynamic size (U.S. Patent Application 2009/0256118 A1 published on 15 Oct. 2009, IPC: H 01B 1/22, C 09D 1/00) are accepted as a prototype. The known method comprises conducting the reaction of an aqueous solution of silver nitrate and a mixture of aqueous solution of iron sulphate (II), FeSO₄, and an aqueous solution of sodium citrate (Na₃C₆H₅O₇), then, the reaction liquid, so obtained, stays with agglomerate silver particles at T between 0° C. and 100° C. to obtain agglomerate silver particles with a larger size of silver particles subject to subsequent control for particle size. Further, the resulting agglomerate silver particles are filtered, and clean water is added to obtain a colloidal solution of silver nanoparticles, the solution having particles 20 to 200 nm in average diameter. Then, the resulting solution is concentrated and washed, and an organic solvent containing dimethyl sulfoxide (DMSO) is further added to produce liquid coating to form a film with silver nanoparticles.

The disadvantage of both the closest method and similar ones is obtaining aqueous colloidal solutions of precious metals in a form unsuitable for being introduced into soft dosage forms or cosmetic products—ointments and creams, since it is not combined with hydrophobic organic components of such forms. The majority of colloidal solutions of precious metals, known in the prior art, do not allow achieving the required biocompatibility level, so their use in the pharmaceutical and cosmetic industry is limited.

The invention is based on the object to produce a biocompatible colloidal solution of silver nanoparticles in a non-aqueous polar solvent in a state suitable for introduction of silver nanoparticles into soft dosage forms and cosmetic products—ointments and creams, and for combination of silver nanoparticles with hydrophobic organic components in ointments and creams by obtaining silver nanoparticles in those media of ointments and creams.

A further object of the invention was to produce a biocompatible colloidal solution of silver nanoparticles in a non-aqueous polar solvent free from oxalate anions, which are products of ascorbic acid conversion reaction, following ascorbic acid interaction with a silver salt and which can cause pain when ointments and creams, utilizing the claimed biocompatible colloidal solution of silver nanoparticles, is applied by using biocompatible reducing agents alternative to ascorbic acid.

This problem is solved, so that a biocompatible colloidal solution of silver nanoparticles in a non-aqueous polar solvent, preferably, in dimethyl sulfoxide comprises, according to the invention, silver nanoparticles, obtained by reducing silver salt, using a biocompatible reductant, which requires an alkaline medium to reduce silver ions to silver nanoparticles [Ag⁰], and such alkaline medium is formed by tetraalkylammonium hydroxide, and ingredients are taken in amounts allowing to obtain nanoparticles with an average size of 12-20 nm, and the resulting colloid solution is adjusted to neutral pH.

A biocompatible reducing agent may comprise ascorbic acid or glycerine or hydrogen peroxide or ethyl alcohol or glucose.

Tetraalkylammonium hydroxide may comprise tetraethylammonium hydroxide or tetraisopropylammonium hydroxide or tetrabutylammonium hydroxide or tetrapentylammonium hydroxide.

Silver salt may comprise silver nitrate (I).

The average size of silver nanoparticles [Ag⁰] may range 12 . . . 20 nm.

Further, the invention is based on the object to provide a method of obtaining a colloidal solution of silver nanoparticles in a non-aqueous polar solvent, suitable for introduction into soft dosage forms and cosmetic products and to interact with hydrophobic organic components in ointments and creams.

This problem is solved, so that in the method of obtaining a colloidal solution of silver nanoparticles in a non-aqueous polar solvent, preferably, in dimethyl sulfoxide, a silver salt, according to the invention, is reduced with biocompatible reducing agent in an alkaline medium by silver salt solution interacting in dimethyl sulfoxide solution with a biocompatible reductant solution, which requires an alkaline medium to reduce silver ions to silver nanoparticles [Ag⁰], and tetraalkylammonium hydroxide in dimethyl sulfoxide, subject to further adjustment of the resulting colloidal solution to neutral pH.

Neutral pH of the resulting colloidal solution may be adjusted by adding organic acid to the resulting colloidal solution.

Ascorbic acid or glycerine or hydrogen peroxide or ethyl alcohol or glucose may be used as a biocompatible reducing agent.

Tetraethylammonium hydroxide or tetraisopropylammonium hydroxide or tetrabutylammonium hydroxide or tetrapentylammonium hydroxide may be used as tetraalkylammonium hydroxide.

Silver nitrate (I) may be used as a silver salt.

Further, optimal molar ratios were found empirically for components in the biocompatible colloidal solution of silver nanoparticles in a non-aqueous polar solvent, produced by the method disclosed herein, specifically:
molar ratio of the amount of silver in the form of nanoparticles to the amount of ascorbic acid and tetraalkylammonium hydroxide may be within 1:1:10,
molar ratio of the amount of silver in the form of nanoparticles to the amount of glycerine and tetraalkylammonium hydroxide may be within 1:1:10,
molar ratio of the amount of silver in the form of nanoparticles to the amount of hydrogen peroxide and tetraalkylammonium hydroxide may be within 1:15 . . . 100:10,
molar ratio of the amount of silver in the form of nanoparticles to the amount of ethyl alcohol and tetraalkylammonium hydroxide may be within 1:5 . . . 8:10,
molar ratio of the amount of silver in the form of nanoparticles to the amount of glucose and tetraalkylammonium hydroxide may be within 1:100 . . . 300:10.

The combination of essential features of the invention and the technical effect achieved, using the invention, brings the following cause-and-effect result.

As it is known in the prior art, the most common method of obtaining highly stable silver colloids (sols) is Turkevich method (J. Kimling, M. Maier, B. Okenve, V. Kotaidis, H. Ballot, and A. Plech, Turkevich Method for Gold Nanoparticle Synthesis Revisited, Fachbereich Physik der Universität Konstanz, Universitätsstr. 10, D-78457 Konstanz, Germany, J. Phys. Chem. B, 2006, 110 (32), pp 15700-15707, DOI: 10.1021/jp061667w, Publication Date: Jul. 21, 2006). In this method, silver nanoparticles are obtained by chemical reduction of silver salt (AgNO₃) with citric or ascorbic acid (AA), which simultaneously act as a stabilizer of nanoparticles to prevent aggregation and consolidation. Interaction of silver salts with ascorbic acid is efficient in alkaline media only. Inorganic alkalis (NaOH, KOH), as conventionally used, are however insoluble in most dispersion media mentioned above.

To solve this problem, the inventors proposed to use an organic basis—tetraalkylammonium hydroxide, which is readily soluble in polar solvents mentioned above, in particular, in dimethyl sulfoxide (DMSO), a non-toxic, commonly used component of various warming pain-relieving ointments and creams, as alkali to reduce gold salt. To this end, the inventors obtained a biocompatible colloidal solution of silver nanoparticles using tetraethylammonium hydroxide or tetraisopropylammonium hydroxide or tetrabutylammonium hydroxide or tetrapentylammonium hydroxide as tetraalkylammonium hydroxide and confirmed that the solution claimed achieved the properties. The inventors, however, assume that obtaining the claimed biocompatible colloidal solution of silver nanoparticles is possible when other known alkyl groups are used in tetraalkylammonium hydroxide provided such groups are readily soluble in polar solvents.

The difference of the proposed method of obtaining a biocompatible colloidal solution of silver nanoparticles from Turkevich method is creating stronger alkaline medium by obtaining a mixture of two components: the first one being alkali based on sodium hydroxide or tetraethylammonium hydroxide etc. and the second one being sodium ascorbate or tetraethylammonium ascorbate (sodium ascorbate—as a mixture of AA and alkali, tetraethylammonium ascorbate—as a mixture of AA and tetraethylammonium hydroxide). As a result, the conditions are now created for obtaining more concentrated solutions of silver nanoparticles. In Turkevich method described in the source above, concentration of silver [Ag⁰] in the form of nanoparticles is 0.0005 mol/L, while the method claimed provides a concentration of [Ag⁰] in the form of nanoparticles up to 0.002 mol/L and a narrower distribution by size, including, in particular, silver [Ag⁰] nanoparticles with an average size within 12 . . . 20 nm. According to the method disclosed herein, after synthesis, the solution is neutralized by adding acetic or citric acid, and this does not change the system characteristics (except for pH value and ionic strength).

Further, while obtaining the claimed biocompatible colloidal solution of silver nanoparticles, the inventors used both ascorbic acid and biocompatible reducing agents alternative to AA, such as, in particular, glycerine, hydrogen peroxide, ethyl alcohol and glucose. Notably, the list of these biocompatible reducing agents is not exhaustive, and other reducing agents may be known to those skilled in the art provided, however, the agents meet the single requirement to achieve the said technical effect—they reduce silver ions to metal in an alkaline medium. For example, there are a number of similar reducing agents, in particular, hydrazine, hydroquinone, formaldehyde, sodium borohydride, etc., however, the similar reducing agents do not meet the biocompatibility requirement and may not be used to obtain solutions of silver NPs to be used in the pharmaceutical or cosmetic industry.

The invention disclosed herein is illustrated by the following examples of obtaining a biocompatible colloidal solution of silver nanoparticles (NPs) using AA as a reductant or using alternative reductants—glycerine, hydrogen peroxide, ethyl alcohol and glucose, and by the following graphic materials, specifically:

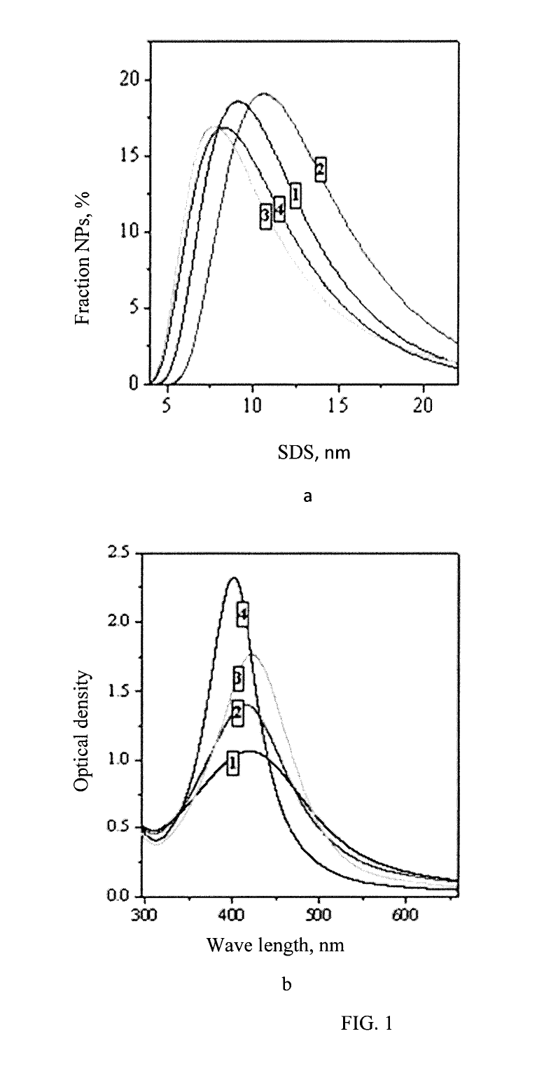

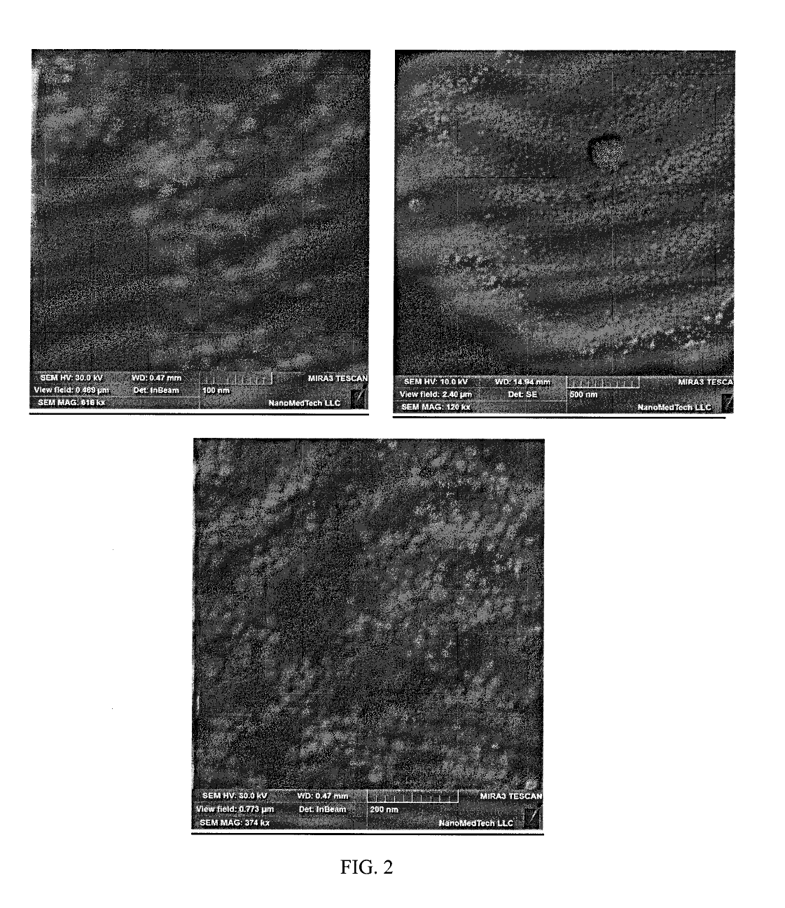

FIG. 1 shows distribution by solvodynamic size (a) and absorption spectra (b) of colloidal silver NPs synthetized with Et₄NOH (curves 1), Pr₄NOH (curves 2), Bu₄NOH (curves 3) and Pt₄NOH (curves 4). [Ag⁰]=2×10⁻³ mol/L, [AA]=1×10⁻³ mol/L, [OH⁻]=1×10⁻² mol/L. Cuvette—1.0 mm (for this and subsequent distributions), DMSO;

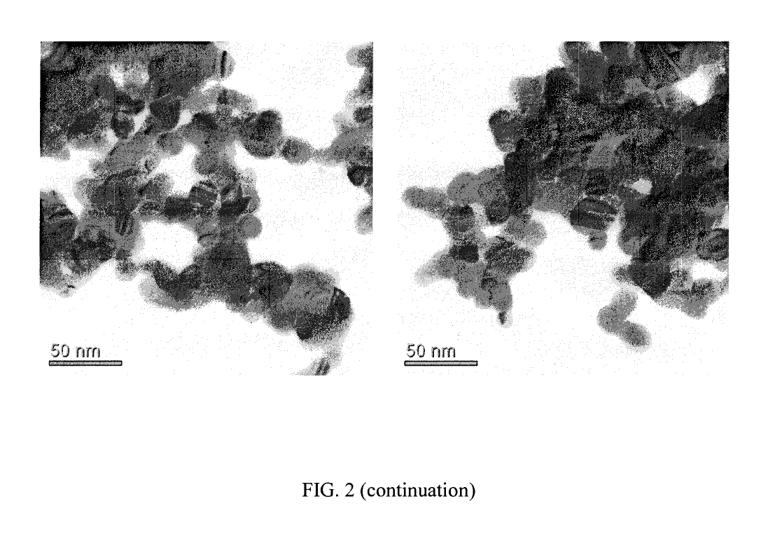

FIG. 2 shows electron microphotographs of colloidal silver NPs synthetized in Example 1;

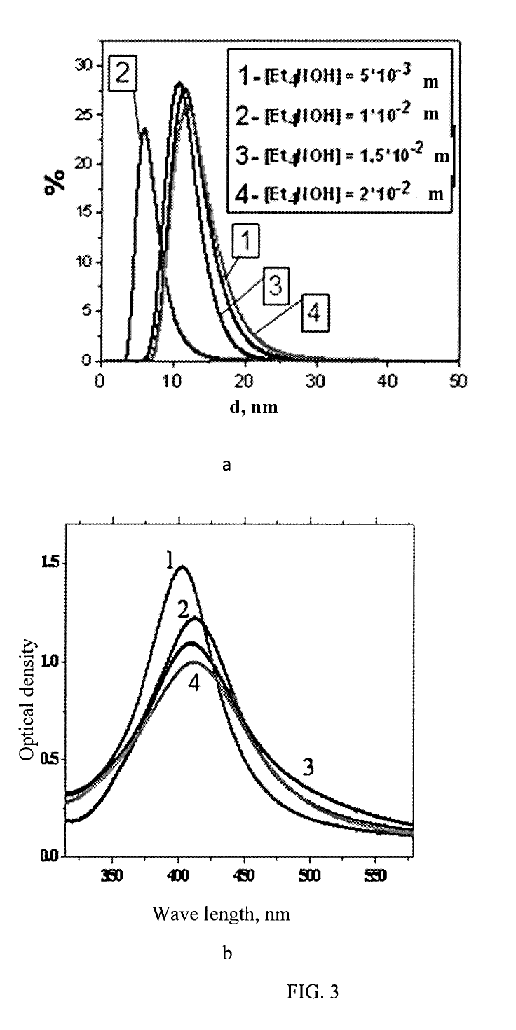

FIG. 3 shows distribution by solvodynamic size (a) and absorption spectra (b) of colloidal silver NPs synthetized with various amounts of Et₄NOH: 5×10⁻³ mol/L (curves 1), 1×10⁻² mol/L (curves 2), 1.5×10⁻² mol/L (curves 3), 2 ×10⁻² mol/L (curves 4). [Ag⁰]=2×10⁻³ mol/L, [AA]=1×10⁻³ mol/L, DMSO;

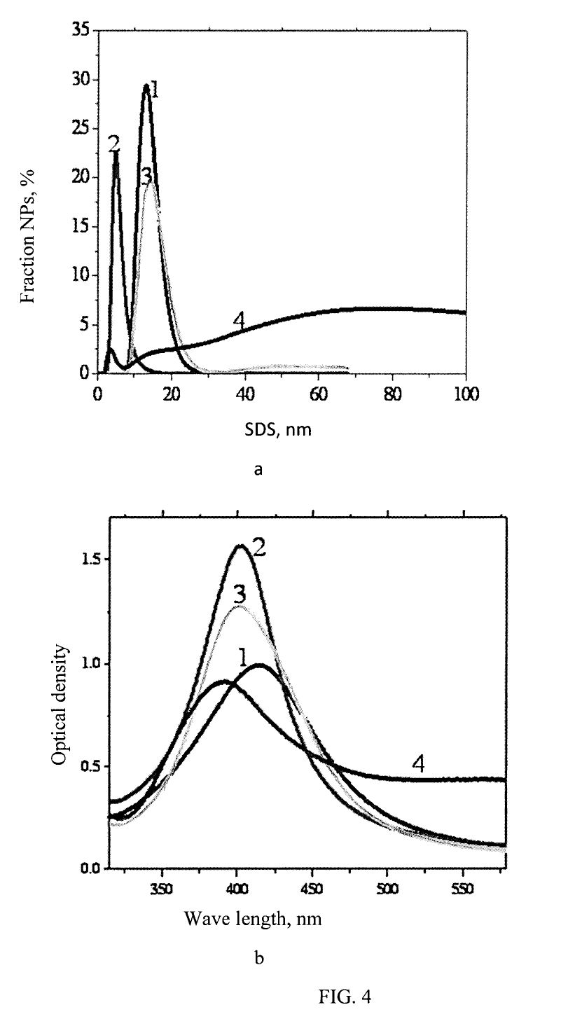

FIG. 4 shows distribution by solvodynamic size (a) and absorption spectra (b) of colloidal silver NPs synthetized with 1×10⁻² mol/L Et₄NOH and various amounts of AA: 5×10⁻⁴ mol/L (curves 1), 1×10⁻³ mol/L (curves 2), 3×10⁻³ mol/L (curves 3), 5×10⁻³ mol/L (curves 4). [Ag⁰]=2×10⁻³ mol/L, DMSO;

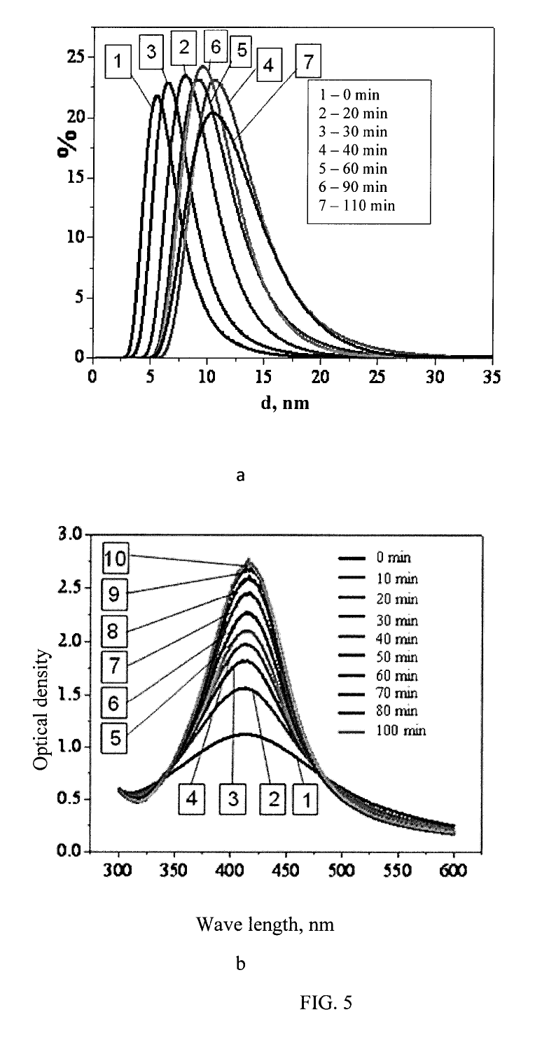

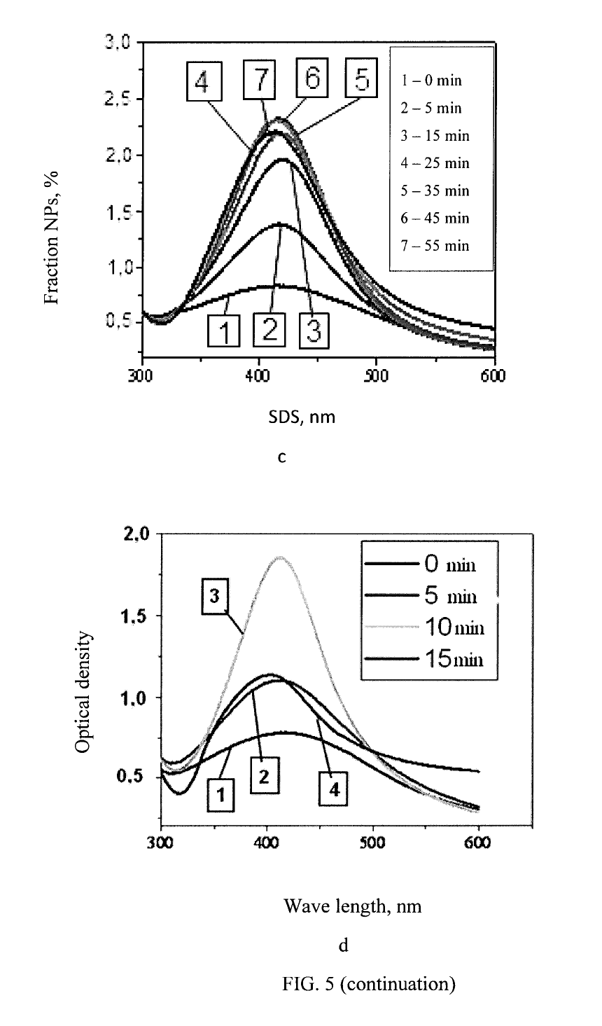

FIG. 5 shows distribution by solvodynamic size (a) and absorption spectra (b) of colloidal silver NPs synthetized at 25° C. and exposed to heating at 50° C. following synthesis. Absorption spectra are given for a colloid heated after synthesis to 70° C. (c) and 90° C. (d). [Ag⁰]=2×10⁻³ mol/L, [AA]=1×10⁻³ mol/L, [Et₄NOH]=1×10⁻² mol/L, DMSO;

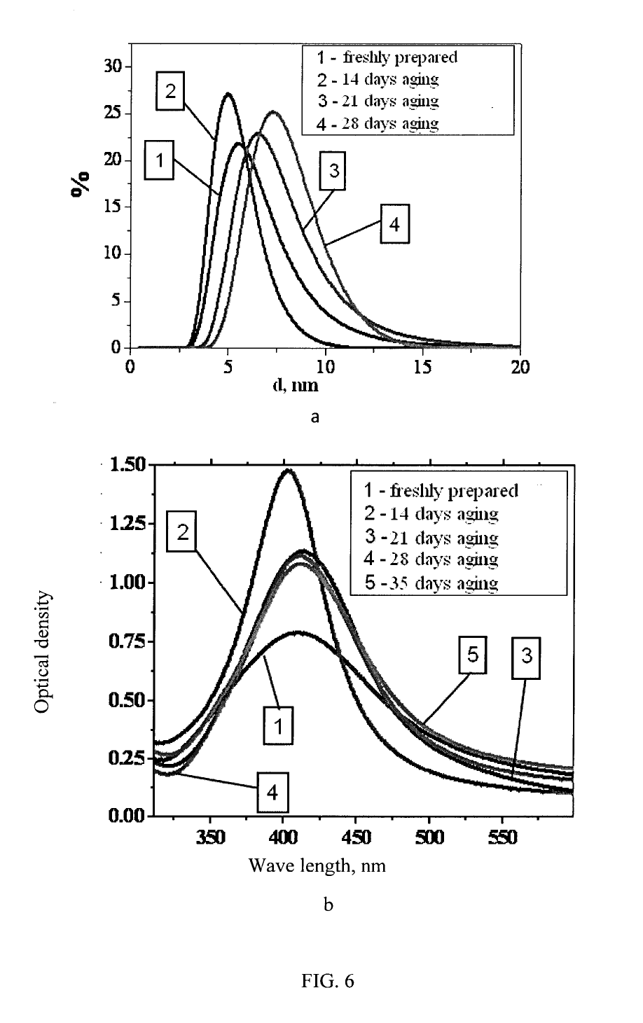

FIG. 6 shows changes in distribution of silver NPs by solvodynamic size (a) and colloid absorption spectra (b) exposed to 25° C. Synthesis is carried out at 25° C. [Ag⁰]=2×10⁻³ mol/L, [AA]=1×10⁻³ mol/L, [Et₄NOH]=1×10⁻² mol/L, DMSO;

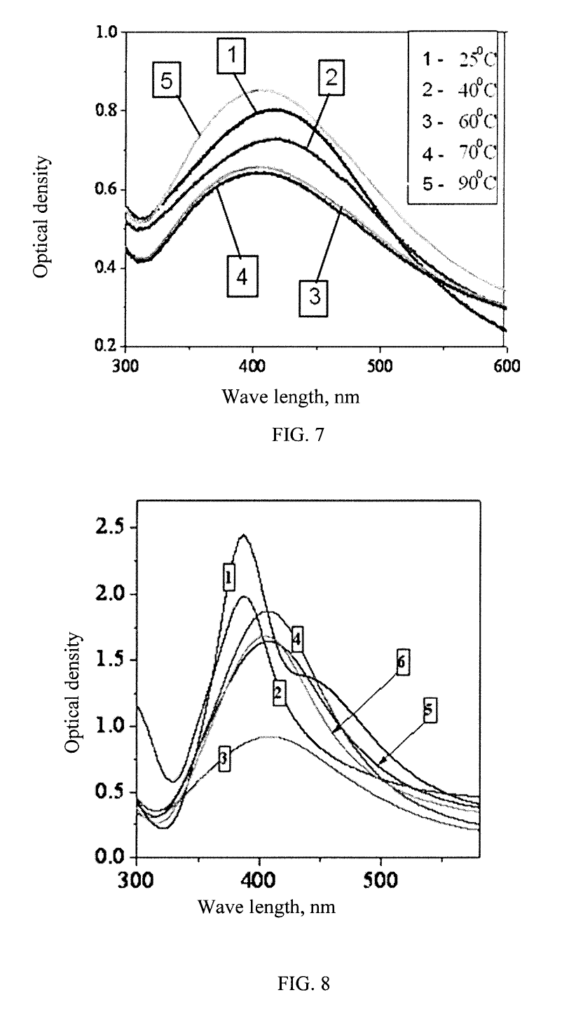

FIG. 7 shows absorption spectra for silver NPs, synthetized at 25° C., 40° C., 60° C., 70° C., 90° C. [Ag⁰]=2×10⁻³ mol/L, [AA]=1×10⁻³ mol/L, [Et₄NOH]=1×10⁻² mol/L, DMSO;

FIG. 8 shows absorption spectra for silver NPs, synthetized in DMSO at 25° C. using 0.5% glycerine (curve 1), 0.1% glucose (curve 2), 0.1% ethanol (curve 3), 0.5% H₂O₂ (curve 4), 0.5% H₂O₂ (curve 5) and 0.75% H₂O₂ (curve 6). [Ag⁰]=2×10⁻³ mol/L, [Et₄NOH]=1×10⁻² mol/L;

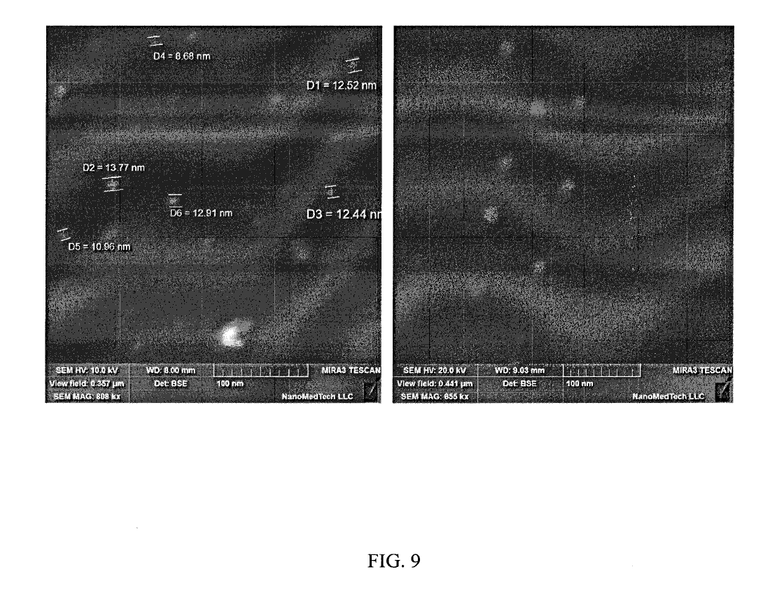

FIG. 9 shows raster electron microphotographs of silver NPs, synthetized in Example No. 21;

Graphic materials, which illustrate the invention disclosed herein, and an example of the resulting biocompatible colloidal solution of silver nanoparticles and the method obtaining thereof are not intended to restrict the scope of claims thereto, but explain the essence of the invention only.

In the first panel of examples, a biocompatible colloidal solution of silver NPs was obtained using ascorbic acid as a reductant and tetraalkylammonium hydroxides, having various alkyl groups, to form an alkaline medium.

Example No. 1: Obtaining a biocompatible colloidal solution of silver NPs using tetraethylammonium hydroxide. Two separate solutions were prepared first as described below. 0.1 ml of 1.0 mol/L aqueous hydroxide tetraethylammonium (Et₄NOH) solution and 0.1 ml of 0.1 mol/L of AA solution in DMSO were added to 4.8 mL of DMSO. Then, 5.0 ml of 0.004 mol/L AgNO₃ solution in DMSO was added under vigorous stirring. This forms a solution of silver NPs containing [Ag⁰]=2×10⁻³ mol/L. Distribution of silver NPs by solvodynamic size (SDS) and colloid absorption spectrum are demonstrated by curves on FIG. 1a and FIG. 1b, respectively.

For this and subsequent panels of examples, a standard magnetic mixer, 300 rpm, was used for stirring. NPs are synthetized at a room temperature in the air.

Example No. 2: Obtaining a biocompatible colloidal solution of silver NPs using tetraisopropylammonium hydroxide. Two separate solutions were prepared first as described below. 0.1 ml of 1.0 mol/L aqueous tetraisopropylammonium hydroxide (Pr₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Distribution of silver NPs by SDS and colloid absorption spectrum are demonstrated by curves 2 on FIG. 1a and FIG. 1b, respectively.

Example No. 3: Obtaining a biocompatible colloidal solution of silver NPs using tetrabutylammonium hydroxide. Two separate solutions were prepared first as described below. 0.1 ml of 1.0 mol/L aqueous tetrabutylammonium hydroxide (Bt₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Distribution of silver NPs by SDS and colloid absorption spectrum are demonstrated by curves 3 on FIG. 1a and FIG. 1b, respectively.

Example No. 4: Obtaining a biocompatible colloidal solution of silver NPs using tetrapentylammonium hydroxide. Two separate solutions were prepared first as described below. 0.1 ml of 1.0 mol/L aqueous tetrapentylammonium hydroxide (Pt₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Distribution of silver NPs by SDS and colloid absorption spectrum are demonstrated by curves 4 on FIG. 1a and FIG. 1b, respectively.

As shown in FIG. 1, distribution of silver NPs, obtained using various organic alkali, and absorption spectra thereof depends on the nature of organic alkali. Silver NPs of smallest size are formed in the presence of tetraethylammonium hydroxide. Therefore, this compound was used as an organic base for subsequent syntheses.

FIG. 2 shows raster (FIG. 1a) and transmission (section 1b) electron microphotographs of Ag NPs, obtained by this method. The data shows, that the average size of NPs is 12-20 nm, that is consistent with findings of dynamic light scattering spectroscopy.

The second panel of examples was intended to select optimal concentration of tetraethylammonium hydroxide to obtain silver NPs, having maximum stability and minimum SDS.

Example No. 5: Two separate solutions were prepared first as described below. 0.05 ml of 1.0 mol/L aqueous tetraethylammonium hydroxide (Et₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 1 on FIG. 3a and FIG. 3b, respectively.

Example No. 6: Two separate solutions were prepared first as described below. 0.1 ml of 1.0 mol/L aqueous tetraethylammonium hydroxide (Et₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 2 on FIG. 3a and FIG. 3b, respectively.

Example No. 7: Two separate solutions were prepared first as described below. 0.15 ml of 1.0 mol/L aqueous tetraethylammonium hydroxide (Et₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 3 on FIG. 3a and FIG. 3b, respectively.

Example No. 8: Two separate solutions were prepared first as described below. 0.2 ml of 1.0 mol/L aqueous tetraethylammonium hydroxide (Et₄NOH) solution and 0.1 ml of 0.1 mol/L AA solution in DMSO were added to 4.7 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 4 on FIG. 3a and FIG. 3b, respectively.

Conclusion: As shown in FIG. 3 and based on findings of the examples described above, stable colloidal solutions of silver NPs with the smallest SDS are produced when 0.01 mol/L tetraethylammonium hydroxide is used.

In the third panel of syntheses, the optimal concentration of reducing agent, ascorbic acid (AA), was selected to produce silver NPs, having maximum stability and minimum SDS.

Example No. 9: Two separate solutions were prepared first as described below. 0.1 ml of 1.0 mol/L aqueous tetraethylammonium hydroxide (Et₄NOH) solution and 0.05 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 1 on FIG. 4a and FIG. 4b, respectively.

Example No. 10: Two separate solutions were prepared first as described below. 0.1 ml of 1.0 mol/L aqueous tetraethylammonium hydroxide (Et₄NOH) solution and 0.1 ml of 0.1 mol/L AA solution in DMSO were added to 4.8 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Distribution of silver NPs by SDS and colloid absorption spectrum are demonstrated by curves 2 on FIG. 4a and FIG. 4b, respectively.

Example No. 11: Two separate solutions were prepared first as described below. 0.1 ml of 1.0 mol/L aqueous tetraethylammonium hydroxide (Et₄NOH) solution and 0.3 ml of 0.1 mol/L AA solution in DMSO were added to 4.6 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Distribution of silver NPs by SDS and colloid absorption spectrum are demonstrated by curves 3 on FIG. 4a and FIG. 4b, respectively.

Example No. 12: Two separate solutions were prepared first as described below. 0.1 ml of 1.0 mol/L aqueous tetraethylammonium hydroxide (Et₄NOH) solution and 0.5 ml of 0.1 mol/L AA solution in DMSO were added to 4.4 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Distribution of silver NPs by SDS and colloid absorption spectrum are demonstrated by curves 3 on FIG. 4a and FIG. 4b, respectively.

Conclusion: As shown in FIG. 4 and based on findings of the examples described above, stable colloidal solutions of silver NPs with the smallest SDS are produced when 1×10⁻³ mol/L ascorbic acid is used.

The fourth panel of examples studied the impact of the temperature of post-synthesis treatment and synthesis temperature on SDS and spectral characteristics of silver NPs.

Example No. 13: Two separate solutions were prepared first as described below. 0.1 ml of 1.0 mol/L aqueous tetraethylammonium hydroxide (Et₄NOH) solution and 0.1 ml of 0.1 mol/L AA solution in DMSO were added to 4.4 mL of DMSO. Then, 5.0 ml of 0.004 mol/L AgNO₃ solution in DMSO was added to this solution under vigorous stirring. Synthesis was carried out at 25° C. This forms a solution of silver NPs, containing [Ag⁰]=2×10⁻³ mol/L.

The solution was further heated at 50° C. for 100 min. Distribution of silver NPs by SDS and colloid absorption spectra during the heating process are demonstrated on FIG. 5a and FIG. 5b, respectively. Similar heating was carried out at 70° C. and 90° C. Colloid absorption spectra during the heating process are demonstrated on FIGS. 5c and 5d, respectively.

Conclusion: As shown in FIG. 5 and based on findings of the examples described above, silver NPs obtained in DMSO at 25° C. practically do not change their average SDS during post-synthesis treatment at 50° C.; spectral data, however, show the progress of the structuring process and crystallization of particles over time, therefore it is further recommended to heat particles for 100 minutes at 50° C. following synthesis. Heating above 50-70° C. results in aggregation of particles and partial coagulation of colloids.

Example No. 14: Two separate solutions were prepared first as described below. 0.1 ml of 1.0 mol/L aqueous tetraethylammonium hydroxide (Et₄NOH) solution and 0.1 ml of 0.1 mol/L AA solution in DMSO were added to 4.4 mL of DMSO. Then, 5.0 ml of 0.004 mol/L AgNO₃ solution in DMSO was added to this solution under vigorous stirring. Synthesis was carried out at 25° C. This forms a solution of silver NPs containing [Ag⁰]=2×10⁻³ mol/L. The solution was held at a room temperature for a month. FIG. 6 show changes in absorption spectra over time.

Conclusion: As shown in FIG. 6 and based on findings of the examples demonstrated above, silver NPs, obtained in DMSO at 25° C. practically do not change their characteristics and retain stable aggregation when held at 25° C. for 1 month.

Example No. 15: Two separate solutions were prepared first as described below. 0.1 ml of 1.0 mol/L aqueous tetraethylammonium hydroxide (Et₄NOH) solution and 0.1 ml of 0.1 mol/L AA solution in DMSO were added to 4.4 mL of DMSO. Then, 5.0 ml of 0.004 mol/L AgNO₃ solution in DMSO was added to this solution under vigorous stirring. Synthesis was carried out at 25° C., 40° C., 60° C., 70° C. and 90° C. This forms a solution of silver NPs, containing [Ag⁰]=2×10⁻³ mol/L. FIG. 7 demonstrate absorption spectra of colloids obtained.

Conclusion: As shown in FIG. 7 and based on findings of the examples described above, silver NPs, obtained in DMSO at above 25° C. form aggregates, as evidenced by increasing light absorbance at 500-600 nm. Therefore, synthesis should be carried out at a temperature below 25° C.

A further panel of experiments was intended to identify opportunities of obtaining silver NPs in DMSO using other biocompatible reducing agents, alternatives to ascorbic acid, in particular, such as glycerine, glucose, hydrogen peroxide and ethanol. Alternative biocompatible reducing agents may be used to relieve short-term pain syndrome associated with the presence of oxalate anion, a product of ascorbic acid oxidation, following intramuscular or intravenous administration of such medicinal product.

A further panel of experiments confirmed the possibility of using glycerine as a reducing agent in the method disclosed herein to produce a biocompatible colloidal solution of silver nanoparticles in a non-aqueous polar solvent.

Example No. 16: Two separate solutions were prepared first as described below. 0.1 ml of 1.0 mol/L aqueous tetraethylammonium hydroxide (Et₄NOH) solution and 0.5 ml of 10% glycerine solution were added to 4.4 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Colloid absorption spectrum is demonstrated by curve 1 on FIG. 8.

The next panel of experiments confirmed the possibility to use glucose as a reducing agent in the method disclosed herein.

Example No. 17: Two separate solutions were prepared first as described below. 0.1 ml of 1.0 mol/L aqueous tetraethylammonium hydroxide (Et₄NOH) solution and 0.1 ml of 10% glucose solution were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Colloid absorption spectrum is demonstrated by curve 2 on FIG. 8.

The next panel of experiments confirmed the possibility to use ethyl alcohol as a reducing agent in the method disclosed herein.

Example No. 18: Two separate solutions were prepared first as described below. 0.1 ml of 1.0 mol/L aqueous tetraethylammonium hydroxide (Et₄NOH) solution and 0.2 ml of 10% ethyl alcohol solution were added to 4.7 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Colloid absorption spectrum is demonstrated by curve 3 on FIG. 8.

The next panel of experiments confirmed the possibility to use hydrogen peroxide, having various concentrations as a reducing agent in the method disclosed herein.

Example No. 19: Two separate solutions were prepared first as described below. 0.1 ml of 1.0 mol/L aqueous tetraethylammonium hydroxide (Et₄NOH) solution and 0.05 ml of 10% H₂O₂ solution were added to 4.8 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Colloid absorption spectrum is demonstrated by curve 4 on FIG. 8.

Example No. 20: Two separate solutions were prepared first as described below. 0.1 ml of 1.0 mol/L aqueous tetraethylammonium hydroxide (Et₄NOH) solution and 0.5 ml of 10% H₂O₂ solution were added to 4.4 mL of DMSO. Then, 5.0 ml of 0.004 mol/L AgNO₃ solution in DMSO was added to this solution under vigorous stirring. This forms a solution of silver NPs containing [Ag⁰]=2×10⁻³ mol/L. Colloid absorption spectrum is demonstrated by curve 5 on FIG. 8.

Example No. 21: Two separate solutions were prepared first as described below. 0.1 ml of 1.0 mol/L aqueous tetraethylammonium hydroxide (Et₄NOH) solution and 0.75 ml of 10% H₂O₂ solution were added to 4.1 mL of DMSO. Then, 5.0 ml of 0.004 mol/L AgNO₃ solution in DMSO was added to this solution under vigorous stirring. This forms a solution of silver NPs, containing [Ag⁰]=2×10⁻³ mol/L. Colloid absorption spectrum is demonstrated by curve 6 on FIG. 8. Electron microphotographs of colloid silver NPs are shown on FIG. 9.

Therefore, the invention disclosed herein allows obtaining a biocompatible colloidal solution of silver nanoparticles in a non-aqueous polar solvent in a form suitable for introduction of silver nanoparticles into soft dosage forms and cosmetic products—ointments and creams, and obtaining a biocompatible colloidal solution of silver nanoparticles in a non-aqueous polar solvent, the use of which avoids pain associated with administration of the product.

1. A biocompatible colloidal solution of silver nanoparticles in a non-aqueous polar solvent, preferably, in dimethyl sulfoxide, characterized in that the solution contains silver nanoparticles, obtained by reducing a silver salt, using a biocompatible reductant, which requires an alkaline medium to reduce silver ions to silver nanoparticles [Ag⁰], and the alkaline medium is obtained with tetraalkylammonium hydroxide, and the ingredients are taken in such amount, that allows obtaining nanoparticles with an average size of 12-20 nm, and the resulting colloidal solution is adjusted to neutral pH.

2. The colloid solution of claim 1, wherein ascorbic agent is a biocompatible reducing agent.

3. The colloid solution of claim 1, wherein glycerine is a biocompatible reducing agent.

4. The colloid solution of claim 1, wherein hydrogen peroxide is a biocompatible reducing agent.

5. The colloid solution of claim 1, wherein ethyl alcohol is a biocompatible reducing agent.

6. The colloid solution of claim 1, wherein glucose is a biocompatible reducing agent.

7. The colloid solution of claim 1, wherein tetraethylammonium hydroxide or tetraisopropylammonium hydroxide or tetrabutylammonium hydroxide or tetrapentylammonium hydroxide is tetraalkylammonium hydroxide.

8. The colloid solution of claim 1, wherein silver nitrate (I) is a silver salt.

9. The colloid solution of claim 1, wherein the average size of silver [Ag⁰] nanoparticles is within 12-20 nm.

10. A method of obtaining a colloidal solution of silver nanoparticles in a nonaqueous polar solvent, preferably, in dimethyl sulfoxide, of claim 1, characterized in that silver salt is reduced by a biocompatible reducing agent in an alkaline medium when the solution of a silver salt and dimethyl sulfoxide interacts with a biocompatible reductant, which requires an alkaline medium to reduce silver ions to silver nanoparticles [Ag⁰], dimethyl sulfoxide and tetraalkyl ammonium hydroxide, and the resulting colloidal solution is then adjusted to neutral pH.

11. The method of claim 10, wherein the resulting colloidal solution is adjusted to neutral pH by adding an organic acid to the resulting colloidal solution.

12. The method of claim 10, wherein ascorbic acid is used as a biocompatible reducing agent.

13. The method of claim 10, wherein glycerine is used as a biocompatible reducing agent.

14. The method of claim 10, wherein hydrogen peroxide is used as a biocompatible reducing agent.

15. The method of claim 10, wherein ethyl alcohol is used as a biocompatible reducing agent.

16. The method of claim 10, wherein glucose is used as a biocompatible reducing agent.

17. The method of claim 10, wherein tetraethylammonium hydroxide or tetraisopropylammonium hydroxide or tetrabutylammonium hydroxide or tetrapentylammonium hydroxide is used as tetraalkylammonium hydroxide.

18. The method of claim 10, wherein silver nitrate (I) is used as a silver salt.
